(19) United States
(12) Patent Application Publication
(10) Pub. No.: US 2009/0060943 A1
(43) Pub. Date: Mar. 5, 2009

(54) Syndrome X Composition and Method of Lowering Blood Pressure and Glycemic Index

(76) Inventors: Brendan Joseph O'Mara, Clare (IE); Ann Marie O'Mara, Limerick (IE)

Correspondence Address: AMIN HALLIHAN, LLC, 444 NORTH ORLEANS STREET, SUITE 400, CHICAGO, IL, 60654, US

(21) Appl. No.: 11/848,277

(22) Filed: Aug. 31, 2007

(51) Int. Cl. A61K 36/02; A61P 3/04; A61P 3/10; A61P 9/12

(52) U.S. Cl. 424/195.17

(57) ABSTRACT

The present invention provides a composition including alginate, seaweed fiber, piperine and potassium citrate for lowering blood pressure, lowering glycemic index, and alleviating conditions associated with Syndrome X. The present invention also provides a method of lowering blood pressure, lowering glycemic index and alleviating conditions associated with Syndrome X. The present invention further provides a method of preventing or protecting against heart disease, diabetes, and hypertension.

### FIELD OF THE INVENTION

The present invention relates to a composition containing alginate used for lowering blood pressure and glycemic index in a body, particularly a human body. The present invention further relates to a composition containing alginate for reducing the conditions associated with Syndrome X. The present composition is designed to be ingested and taken as a beverage.

### BACKGROUND OF THE INVENTION

Syndrome X is a group of interrelated conditions, disorders and associated symptoms. Syndrome X is estimated to affect 1 in 3 North Americans. Syndrome X includes insulin resistance, abnormal blood fats (high triglycerides levels, decreased HDL), obesity and being overweight, and high blood pressure. These conditions further include hypertriglyceridemia (high blood lipid); low HDL-cholesterol; hyperinsulinemia (high blood insulin); hyperglycemia (high blood glucose); and hypertension (high blood pressure). Syndrome X has been found to predispose for diabetes, hypertension and heart disease.

For example, as illustration of the interrelatedness of Syndrome X conditions, insulin resistance is the inability to properly respond to the intake of dietary carbohydrates and sugars resulting in elevated glucose levels in the blood. Insulin is a hormone which processes glucose in the body by binding to receptors on cells and facilitating the passage of glucose from the blood to the cell. If the body is resistant to insulin then the insulin does not facilitate the passage of glucose which results in elevated glucose levels in the blood. The pancreas releases more insulin to achieve a normal amount of glucose in the blood and if the cells do not respond to this additional insulin then high blood glucose levels or Type II diabetes can result. Insulin resistance further causes an imbalance in blood lipids, such as high triglycerides and low HDL which are risk factors for heart disease. Also, eating large amounts of dietary carbohydrates, including sweets, pastas and breads, can raise cholesterol, triglycerides, and insulin levels associated with Syndrome X. Further elevated insulin levels leads to obesity and high blood pressure.

Syndrome X is established in a human by the presence of certain waist circumference, triglyceride levels, HDL cholesterol levels, blood pressure, and fasting glucose levels. Abdominal obesity or a waist circumference for men greater than 102 cm (40 in) and for women greater than 88 cm (35 in) is an indication of Syndrome X. Triglyceride levels of greater than or equal to 150 mg/dl is also an indication of Syndrome X. Syndrome X is also associated with levels of HDL cholesterol of less than 40 mg/dl for men and 50 mg/dl for women. Blood pressures of greater than or equal to 130 mm Hg systolic or 85 mm Hg diastolic or current use of blood pressure medication are also indications of Syndrome X. Finally, a fasting glucose level of greater than or equal to 110 mg/dl or current use of a diabetes medication are indications of Syndrome X.

Currently Syndrome X conditions can be reduced by exercising which leads to weight loss, higher HDL cholesterol levels, lower blood pressure and lower insulin resistance. Syndrome X conditions can also be alleviated by reducing alcohol and refined carbohydrate intake which leads to lower triglyceride levels. However, changing diet and/or exercise habits can be very difficult. Additionally, it is difficult to treat Syndrome X and all of its interrelated conditions with one ingredient. Accordingly, a natural, effective ingested composition having components which alleviate the conditions associated with Syndrome X has been developed.

### SUMMARY OF THE INVENTION

One object of the composition of the present invention is for a natural, effective ingested composition having components which alleviate the conditions associated with Syndrome X.

A more specific object of the present invention is to overcome one or more of the problems discussed above.

Another object of the present invention is for a composition having the ability to lower blood pressure, lower glycemic index, reduce insulin resistance and reduce the amount of insulin needed by the body.

A further object of the present invention is for a composition which reduces or alleviates the conditions associated with Syndrome X without a change in diet or exercise.

A still further object of the present invention is for a composition having a tasteful flavor.

An even further object of the present invention is for a composition which prevents or protects against heart disease, diabetes, and hypertension.

Still another object of the present invention is for a composition which increases the bioavailability of the components.

In accordance with the present invention, a composition containing alginate for lowering blood pressure and glycemic index, and alleviating the conditions associated with Syndrome X in the human body has been developed. In one form, the composition has been developed as a beverage.

The composition of the present invention includes alginate, seaweed fiber, piperine, and potassium citrate that has been developed specifically for the purpose of lowering blood pressure, lowering glycemic index and alleviating conditions associated with Syndrome X. The composition of the present invention also is believed to reduce insulin resistance, reduces insulin requirements, and protects against or prevents heart disease, diabetes, and hypertension.

In one embodiment, the composition may include and/or consist of about 0.01%-10% (w/v) alginate, about 0.01%-10% (w/v) seaweed fiber, about 0.0002% to 1% (w/v) piperine and about 0.008% to 1.2% (w/v) potassium citrate, in water.

The present invention also relates to a method of lowering blood pressure, lowering glycemic index and alleviating the conditions associated with Syndrome X. The method includes administering to a human a composition comprising alginate, seaweed fiber, piperine and potassium citrate, and the components lowering the blood pressure and glycemic index, and alleviating the conditions associated with Syndrome X in the body.

These and other embodiments are more fully described in connection with the detailed description.

### DETAILED DESCRIPTION OF THE INVENTION

The present invention relates to a composition containing alginate used for lowering blood pressure, lowering glycemic index, and alleviating the conditions associated with Syndrome X. The present invention further relates to a composition containing alginate used to reduce insulin resistance, reduce insulin requirements, and prevent and protect against heart disease, diabetes, and hypertension in humans. In one form, the composition is designed to be ingested and taken as a beverage.

One embodiment of the present invention relates to a composition having alginate, seaweed fiber, piperine and potassium citrate.

The components of the composition of the present invention lower blood pressure and glycemic index and alleviate the conditions associated with Syndrome X in the human body. The composition of the present invention also reduces insulin resistance, reduces insulin requirements, and prevents or protects against heart disease, diabetes, and hypertension in humans.

The term “alginate” will be defined to include alginate, alginic acid and its various forms, and other salts of alginic acid.

Alginate is produced by seaweed. Alginate has the ability to form a gel when its various polymer strands are cross-linked with divalent or trivalent cations. When alginate comes in contact with hydrogen ions and divalent cations, such as calcium, it forms a gel. When ingested, alginate forms a gel in the stomach which is believed to produce an immediate and sustained effect of satiety and reduces food intake. Further, the alginate reduces the activity of certain digestive enzymes, such as pepsin and trypsin. This effect reduces the glycemic load of food taken in and reduces the amount of insulin required by the body.

The known health benefits of dietary fiber alone include lowering the risk of colon cancer, heart disease, lowering serum lipids, and improving glucose tolerance due to the high water-holding capacity, viscosity enhancing effect, gel forming properties, high adsorption capacity including cation exchange, activation of intestinal enzymes, and alteration of microflora of the gut. Dietary fibers also have great adsorption potency for less polar compounds. Further, dietary fibers have a great binding capacity for various types of bile salts.

Additionally, alginate can block the absorption of metal ions, cholesterol and glucose by binding wherein the levels of total cholesterol, blood glucose, and insulin are reduced. Alginate has the ability to bind to metals in this manner. Further alginates contain guluronic acid and an increase in guluronic acid increases the affinity of alginates for divalent cations such as Pb²⁺, Cu²⁺, Cd²⁺, Zn²⁺, and Ca²⁺. The intake of dietary alginates alone results in a number of potentially beneficial physiological effects, such as reduced intestinal absorption, increased satiety, reduced damaging potential GI luminal contents, modulation of colonic microflora, and elevation of colonic barrier function.

In one embodiment of the present invention, the composition can include about 0.01% to 10% (w/v) alginate. In a further embodiment of the present invention, the composition can include about 0.5% to 6% (w/v) alginate. The composition can still further include about 0.1% to 1.5% (w/v) alginate. In an even further embodiment of the present invention, the composition can include at least about 0.1% (w/v) alginate.

In one embodiment of the present invention, the composition can include about 0.05 g to 50 g alginate in 500 mL of water. In a further embodiment of the present invention, the composition can include about 0.25 g to 30 g alginate in 500 mL of water. The composition can still further include about 0.5 g to 7.5 g alginate in 500 mL of water. In an even further embodiment of the present invention, the composition can include at least about 0.5 g alginate in 500 mL of water.

In a further embodiment of the present invention, the composition can include at least about 0.5 g alginate.

Seaweed fiber has amylase inhibitory effects. Amylase is a type of enzyme responsible for breaking down starch into glucose. An amylase inhibitor taken before a meal reduces the glycemic load of a meal.

In one embodiment of the present invention, the composition can include about 0.01% to 10% (w/v) seaweed fiber. In a further embodiment of the present invention, the composition can include about 0.5% to 6% (w/v) seaweed fiber. The composition can still further include about 0.2% to 2% (w/v) seaweed fiber. In an even further embodiment of the present invention, the composition can include at least about 0.2% (w/v) seaweed fiber.

In one embodiment of the present invention, the composition can include about 0.05 g to 50 g seaweed fiber in 500 mL of water. In a further embodiment of the present invention, the composition can include about 0.25 g to 30 g seaweed fiber in 500 mL of water. The composition can still further include about 1 g to 10 g seaweed fiber in 500 mL of water. In an even further embodiment of the present invention, the composition can include at least about 1 g seaweed fiber.

In a further embodiment of the present invention, the composition can include at least about 1 g seaweed fiber.

Piperine can increase the bioavailability of the portion of potassium citrate not trapped during the alginate gel formation wherein the absorption of the potassium citrate which has a blood pressure lowering effect can increase. Thus the piperine can negate the loss of the potassium citrate trapped in the alginate gel formation.

Piperine does not generally dissolve in water. Suitably, the piperine of the present invention can be bound to dextrin wherein the piperine/dextrin complex dissolves in water when the composition is taken as a beverage.

In one embodiment of the present invention, the composition can include about 0.0002% to 1% (w/v) piperine. In a further embodiment of the present invention, the composition can include about 0.0006% to 0.4% (w/v) piperine. The composition can still further include about 0.001% to 0.006% (w/v) piperine. In an even further embodiment of the present invention, the composition can include about at least 0.001% (w/v) piperine.

In another embodiment of the present invention, the composition can include about 1 mg to 5 g piperine in 500 mL of water. In another embodiment of the present invention, the composition can include about 3 mg to 2 g piperine in 500 mL of water. The composition can still further include about 5 mg to 30 mg piperine in 500 mL of water. In an even further embodiment of the present invention, the composition can include about at least 5 mg piperine in 500 mL of water.

In a further embodiment of the present invention, the composition can include at least about 5 mg piperine.

Potassium citrate acts to maintain the gel formed by the alginate in the stomach longer and increases the satiety effect of the alginate. The potassium citrate makes the gel formed by the alginate more elastic and less prone to being fractured. Therefore, the amount of food intake and the glycemic load is reduced. The amount of insulin required by the body is also reduced when the glycemic load of a meal is reduced.

In one embodiment of the present invention, the composition can include about 0.008% to 1.2% (w/v) potassium citrate. In a further embodiment of the present invention, the composition can include about 0.03% to 0.6% (w/v) potassium citrate. The composition can still further include about 0.04% to 0.108% (w/v) potassium citrate. In an even further embodiment of the present invention, the composition can include at least about 0.036% (w/v) potassium citrate.

In one embodiment of the present invention, the composition can include about 40 mg to 6 g potassium citrate in 500 mL of water. In a further embodiment of the present invention, the composition can include about 150 mg to 3 g potassium citrate in 500 mL of water. The composition can still further include about 200 mg to 540 mg potassium citrate in 500 mL of water. In an even further embodiment of the present invention, the composition can include at least about 180 mg potassium citrate.

In a further embodiment of the present invention, the composition can include at least about 180 mg potassium citrate.

The alginate, seaweed fiber, piperine and potassium citrate of the present invention work synergistically to lower blood pressure, lower glycemic index, and alleviate the conditions associated with Syndrome X. The present invention provides satiety and reduces food intake. The present invention reduces insulin requirements, and reduces insulin resistance. The present invention protects against and prevents heart disease, diabetes, and hypertension.

The alginate contains a high amount of guluronic acid and reduces the activity of digestive enzymes such as pepsin and trypsin reducing the glycemic load of food and lowers the insulin response. The seaweed fiber works together with the alginate by inhibiting amylase activity which also reduces the glycemic load of food. Also, the seaweed fiber, which has ion exchange properties, works together with the piperine and potassium citrate to scavenge sodium ions and exchange them for potassium, which reduces blood systolic pressure. The composition components also work together to bind and eliminate bile salts, which lowers cholesterol.

One embodiment of the present invention relates to a composition having about 0.01%-10% (w/v) alginate, about 0.01%-10% (w/v) seaweed fiber, about 0.0002% to 1% (w/v) piperine, and about 0.008% to 1.2% (w/v) potassium citrate.

The following example of one embodiment of the present invention provides a composition in about 500 ml of water and demonstrates the scope of the present invention. The composition includes:

about 0.5 g alginate;

about 1 g seaweed fiber;

about 5 mg piperine; and

about 180 mg potassium citrate.

Another example of one embodiment of the present invention provides a composition in about 500 ml of water and demonstrates the scope of the present invention. The composition includes:

about 0.1% (w/v) alginate;

about 0.2% (w/v) seaweed fiber;

about 0.001% (w/v) piperine; and

about 0.036% (w/v) potassium citrate.

A further example of one embodiment of the present invention provides a composition in water and demonstrates the scope of the present invention. The composition includes:

about 0.5 g alginate;

about 1 g seaweed fiber;

about 5 mg piperine; and

about 180 mg potassium citrate.

In another embodiment, the ratio of alginate, seaweed fiber and potassium citrate is 1:2:0.7.

In another embodiment, the alginate can also be high in guluronic acid. In another embodiment, the alginate can have 50-100% guluronic acid.

In another embodiment, the seaweed fiber can be from Ascophyllum nodosum.

In another embodiment, the potassium citrate combined with seaweed fiber reduces blood pressure by scavenging for sodium ions in exchange for potassium.

In another embodiment, the alginate can be sodium alginate.

In another embodiment, the composition can include a flavor component, a sweetener component and/or combinations thereof for improved taste.

In another embodiment, the composition can be further sterilized and have an extended shelf-life through an ultra-high temperature process. The ultra-high temperature process includes heating the composition for about 2-3 seconds at a temperature of at least approximately 275 degrees Fahrenheit. The ultra-high temperature process aids in killing microbes present in the composition prior to mixing in the water.

In one embodiment of the present invention, the composition can be a beverage. Suitably, the composition can have a flavor and can be consumed as a snack. In another embodiment of the present invention, the composition can be in the form of a pill, a capsule, a powder, a chewable, or syrup. In another embodiment, the composition can be in a cereal bar that does not contain calcium or acid for flavoring.

In another embodiment, the composition can include a preservative. The preservative can include parabens and can further include methyl and propyl parabens. Parabens are common preservatives in the cosmetic, food and beverage, and pharmaceutical industries due to their anti-microbial effects. In the U.S., parabens are GRAS approved and allowed at a maximum concentration of 0.1%. In a further embodiment, the composition can include methyl and propyl parabens in a 3:1 or 2:1 ratio. In a further embodiment, the composition can include 0.375 g of methyl paraben and 0.125 g of propyl paraben.

The present invention also relates to a method of lowering blood pressure, lowering glycemic index and alleviating the conditions associated with Syndrome X. The method includes administering a composition comprising alginate, seaweed fiber, piperine and potassium citrate, and the components lowering the blood pressure and glycemic index, and alleviating the conditions associated with Syndrome X in the body. The method can include providing satiety and reducing food intake. The method can include reducing insulin requirements, and reducing insulin resistance. The method can further include protecting against and preventing heart disease, diabetes, and hypertension.

While in the foregoing specification this invention has been described in relation to certain preferred embodiments thereof, and many details have been set forth for the purpose of illustration, it will be apparent to those skilled in the art that the invention is susceptible to additional embodiments and that certain details described herein can be varied considerably without departing from the basic principles of the invention.

We claim:

1. A composition for alleviating conditions associated with Syndrome X, comprising:
alginate;
seaweed fiber;
piperine; and
potassium citrate.

2. The composition of claim 1 wherein the composition is in a beverage form.

3. The composition of claim 1 wherein the alginate is sodium alginate.

4. The composition of claim 1 wherein the ratio of alginate to seaweed fiber to potassium citrate is between 1:2:.07.

5. The composition of claim 1 wherein the alginate comprises 50-100% of guluronic acid.

6. The composition of claim 1 wherein the seaweed fiber is Ascophyllum nodosum.

7. The composition of claim 1 further comprising a flavoring component, a sweetening component and combinations thereof.

8. The composition of claim 1 wherein the alginate is 0.01% to 10% (w/v), the seaweed fiber is 0.01% to 10% (w/v), the piperine is about 0.0002% to ‘% (w/v), and the potassium citrate is 0.008% to 1.2% (w/v), in water.

9. The composition of claim 1 wherein the alginate is 0.05 g to 50 g, the seaweed fiber is 0.05 g to 50 g, the piperine is about 1 mg to 5 g, and the potassium citrate is 40 mg to 6 g, in water.

10. The composition of claim 1 wherein the alginate is 0.5 g, the seaweed fiber is 1 g, piperine is about 5 mg, and the potassium citrate is 180 mg, in water.

11. The composition of claim 1 wherein the piperine is in the form of a piperine/dextrin complex.

12. A composition for alleviating conditions associated with Syndrome X, comprising:
at least about 0.10% (w/v) alginate;
at least about 0.20% (w/v) seaweed fiber;
at least about 0.001% (w/v) piperine; and
at least about 0.036% (w/v) potassium citrate,
in about 500 mL of water.

13. A method for alleviating conditions associated with Syndrome X in the human body comprising the steps of:
administering the composition in accordance with claim 1; and
wherein the administered composition acts to lower blood pressure, lower glycemic index, and alleviate conditions associated with Syndrome X present prior to administering the composition.

14. The method of claim 13 further comprising reducing insulin resistance and reducing the amount of insulin required by the body.

15. The method of claim 13 further comprising providing satiety and reducing food intake.

16. A method of preventing heart disease, diabetes and hypertension comprising the steps of:
administering the composition in accordance with claim 1;
wherein the administered composition acts to lower blood pressure, lower glycemic index, and alleviate conditions associated with Syndrome X present in prior to administering the composition; and
wherein the administered composition acts to prevent heart disease, diabetes, and hypertension.

17. The method of claim 16 further comprising reducing insulin resistance and reducing the amount of insulin required by the body.

18. The method of claim 16 further comprising providing satiety and reducing food intake.

19. The composition of claim 1 further comprising calcium.

20. The composition of claim 19 wherein the alginate cross-links with the calcium to provide a beverage having a gelled consistency.
